(19) United States
(12) Patent Application Publication
(10) Pub. No.: US 2026/0077160 A1
(43) Pub. Date: Mar. 19, 2026

(54) Catheter Tip

(71) Applicant: TERUMO KABUSHIKI KAISHA, Tokyo (JP)

(72) Inventors: Kotaro Kaneko, Tokyo (JP); Ryota Matsuzaki, Yokohama-shi (JP)

(73) Assignee: TERUMO KABUSHIKI KAISHA, Tokyo (JP)

(21) Appl. No.: 19/325,078

(22) Filed: Sep. 10, 2025

(30) Foreign Application Priority Data
Sep. 17, 2024 (JP) 2024-160226

(51) Int. Cl. A61M 25/00; A61M 25/06

(52) U.S. Cl. CPC A61M 25/008; A61M 25/0045; A61M 25/0068; A61M 25/0662; A61M 2025/0059; A61M 2205/0238

(57) ABSTRACT

A catheter tip contains a catheter tip resin layer containing a resin having an ester bond and/or an amide bond, carbodiimide, and tungsten carbide (WC). The content of the carbodiimide is more than 0% by mass and less than 0.5% by mass with respect to the total mass of the catheter tip resin layer. An elongation residual ratio is 70% or more.

### CROSS-REFERENCES TO RELATED APPLICATIONS

This application is based on and claims priority to Japanese Patent Application No. 2024-160226 filed on Sep. 17, 2024, the entire content of which is incorporated herein by reference.

### TECHNOLOGICAL FIELD

The present disclosure relates to a catheter tip.

### BACKGROUND DISCUSSION

When the treatment or diagnosis of a stenosed part is performed, a catheter is used to insert a medical device into a living body and introduce the medical device into a target site. In order to accurately grasp the position of the distal end of the catheter when the catheter is inserted into the living body, the distal portion (catheter tip) of the catheter usually contains a radiopaque substance that enables detection with X-rays. For example, a metal tungsten powder is known as an excellent radiopaque substance and is used as the material of a catheter tip (for example, U.S. Pat. No. 5,584,821). As the material of the catheter, a resin having an amide bond or an ester bond is generally used.

The catheter is not only used immediately, but may be stored for a long period of time before use. A resin is usually used for the catheter tip for moldability and flexibility, but when tungsten is blended in the catheter tip as a radiopaque substance, the resin may deteriorate.

### SUMMARY

A catheter tip is disclosed that is less likely to degrade a resin even when stored for a long period of time, and a catheter including the catheter tip.

(1) A catheter tip comprising a catheter tip resin layer containing a resin having an ester bond and/or an amide bond, carbodiimide, and tungsten carbide (WC), wherein a content of the carbodiimide is more than 0% by mass and less than 0.5% by mass with respect to a total mass of the catheter tip resin layer.

(2) The catheter tip according to (1), wherein an elongation residual ratio represented by the following Formula (1) is 70% or more:

$$Elongation residual ratio [ \% ] = B / A \times 100 \qquad Formula ( 1 )$$

wherein A represents a breaking stroke length in a tensile test of the catheter tip resin layer before a load test, B represents a breaking stroke length in the tensile test of the catheter tip resin layer after the load test, and the load test is performed by allowing a dumbbell-shaped test piece to stand for 30 hours under conditions of a temperature of 80° C. and a humidity of 95% RH.

(3) The catheter tip according to (1) or (2), wherein a content of the carbodiimide is more than 0% by mass and 0.3% by mass or less with respect to the total mass of the catheter tip resin layer.

(4) The catheter tip according to any one of (1) to (3), wherein the carbodiimide is composed of only one kind.

(5) The catheter tip according to any one of (1) to (4), wherein a content of the tungsten carbide is 60% by mass or more with respect to the total mass of the catheter tip resin layer.

(6) The catheter tip according to any one of (1) to (5), wherein a content of a hindered amine light stabilizer (HALS) is more than 0% by mass and 0.1% by mass or less with respect to the total mass of the catheter tip resin layer.

(7) The catheter tip according to any one of (1) to (6), wherein the elongation residual ratio is 80% or more.

(8) The catheter tip according to claim 1, wherein a strength residual ratio represented by the following Formula (2) is 60% or more:

$$Strength residual ratio [ \% ] = D / C \times 100 \qquad Formula ( 2 )$$

wherein C represents a breaking strength in a tensile test of the catheter tip resin layer before the load test, and D represents a breaking strength in the tensile test of the catheter tip resin layer after the load test.

(9) A catheter including the catheter tip according to any one of (1) to (8).

(10) The catheter according to (9), wherein the catheter is used as at least one of a guiding catheter and a guiding sheath.

(11) A catheter comprising: a catheter body portion extending in an axial direction; a catheter tip disposed on a distal side of the catheter body portion; a hub disposed on a proximal side of the catheter body portion; and wherein the catheter includes a catheter tip resin layer containing a resin having an ester bond and/or an amide bond, carbodiimide, and tungsten carbide, wherein a content of the carbodiimide is more than 0% by mass and less than 0.5% by mass with respect to a total mass of the catheter tip resin layer.

(12) A catheter comprising: a catheter body portion extending in an axial direction; a catheter tip disposed on a distal side of the catheter body portion; the catheter includes a catheter tip resin layer containing a resin having an ester bond and/or an amide bond, carbodiimide, and tungsten carbide, wherein a content of the carbodiimide is more than 0% by mass and less than 0.5% by mass with respect to a total mass of the catheter tip resin layer; wherein the catheter tip has an elongation residual ratio represented by Formula (1) is 70% or more:

$$e longation residual ratio [ \% ] = B / A \times 100 \qquad Formula ( 1 )$$

wherein A represents a breaking stroke length in a tensile test of the catheter tip resin layer before a load test, B represents a breaking stroke length in the tensile test of the catheter tip resin layer after a load test, and the load test is performed by allowing a dumbbell-shaped test piece to stand for 30 hours under conditions of a temperature of 80° C. and a humidity of 95% RH; and wherein the content of the carbodiimide in the catheter tip is more than 0% by mass and 0.3% by mass or less with respect to the total mass of the catheter tip resin layer.

According to the present disclosure, a catheter tip is provided that is less likely to degrade a resin even when stored for a long period of time, and a catheter including the catheter tip.

### BRIEF DESCRIPTION OF THE DRAWINGS

FIG. 1 is a diagram illustrating the overall configuration of a catheter.

FIG. 2 is a schematic cross-sectional view in the vicinity of bonding of a catheter tip and a catheter body portion.

### DETAILED DESCRIPTION

The present disclosure provides a catheter tip including a catheter tip resin layer containing a resin having an ester bond and/or an amide bond, carbodiimide, and tungsten carbide (WC), wherein a content of the carbodiimide is more than 0% by mass and less than 0.5% by mass with respect to a total mass of the catheter tip resin layer.

A metal tungsten powder is usually used in combination with a resin as a radiopaque substance in a catheter tip. The present inventors have inferred that when the cause of the deterioration of the resin due to long-term storage is the coexistence of metal tungsten, the hydrolysis of ester bond and/or amide bond sites is promoted. That is, when metal tungsten is present in an air atmosphere containing moisture, a chemical reaction represented by the following Formula 3 proceeds, and thus hydrogen ions (that is, ions represented by “H⁺”) are released.

$$W + ( 3 / 2 ) {O}_{2} \to {WO}_{3} \qquad ( Formula 3 )$$

$${WO}_{3} + {H}_{2} O \to {H}_{2} {WO}_{4} \to 2 {H}^{+} + {WO}_{4}^{2 -}$$

The hydrogen ions promote the hydrolysis of the ester bond and/or amide bond sites. Furthermore, as the hydrolysis proceeds, the terminal carboxyl group of a degradation product acts as an acid, and the reaction rate of the hydrolysis can be increased. Therefore, it is considered that the hydrolysis of the resin gradually proceeds even under a normal temperature and humid environment due to the long-term storage of a catheter, and as a result, the resin may deteriorate over time.

Based on such findings, the present inventors have extensively conducted studies, and resultantly found that when tungsten carbide (WC) is used as the radiopaque substance in place of the metal tungsten (W) powder, the deterioration of the resin under high temperature and high humidity load conditions is suppressed. It is considered that by using tungsten carbide (WC), the generation of tungsten oxide (WO₃) as shown in Formula 3 is suppressed, so that the subsequent hydration reaction of tungsten oxide is also less likely to proceed, and the generation of acidic hydrogen ions is suppressed, so that a decrease in a pH value is suppressed, and as a result, the hydrolysis of the ester bond and/or the amide bond can be suppressed from proceeding.

As described above, by using the tungsten carbide as the radiopaque substance and further adding carbodiimide as a hydrolysis inhibitor to the layer in which the resin is present, it is considered that the progress of the hydrolysis is further suppressed, and as a result, the deterioration of the resin is remarkably suppressed. Note that the above mechanism is based on presumption, and correctness or incorrectness does not affect the technical scope of the present disclosure.

Hereinafter, a catheter according to the present disclosure will be described with reference to the drawings. In the description of the drawings, the same elements are denoted by the same reference numerals, and redundant description will be omitted. A dimensional ratio in each drawing is exaggerated for convenience of description, and may be different from an actual ratio.

Furthermore, a form in which two or three or more of preferable individual forms of the present disclosure described below are combined is also regarded as a preferable form of the present disclosure and disclosed in the present specification (that is, it is a legitimate basis for amendments).

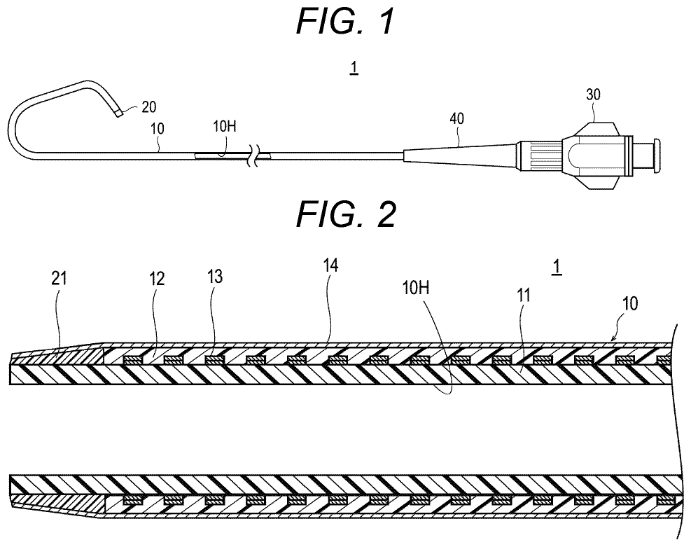

FIG. 1 is a diagram illustrating the overall configuration of a catheter 1 of the present disclosure. In FIG. 1, the part (left side in FIG. 1) of the catheter 1 inserted into a living body is referred to as a distal side, the part of the catheter 1 on which a hub 30 is disposed is referred to as a proximal side, and the direction in which a catheter body portion 10 of the catheter 1 extends is referred to as an axial direction. A direction away from or approaching the catheter body portion 10 in the transverse cross section (cross section orthogonal to the axis) of the catheter body portion 10 with the axial direction of the catheter body portion 10 as a reference axis is referred to as a “radial direction”.

The catheter 1 includes the catheter body portion 10 extending in the axial direction, a catheter tip 20 disposed on the distal side of the catheter body portion 10, the hub 30 disposed on the proximal side of the catheter body portion 10, and a kink-resistant protector (strain relief) 40 disposed between the catheter body portion 10 and the hub 30. The distal portion of the catheter body portion 10 and the catheter tip 20 may be joined to each other, and can be joined and integrated by thermal fusion bonding, for example.

FIG. 2 is an enlarged cross-sectional schematic view of the distal structure of the catheter 1 according to an embodiment of the present disclosure. As illustrated in FIG. 2, the catheter 1 is configured as a tubular member having flexibility in which a lumen extending in the axial direction, a distal opening communicating with the lumen, and a proximal opening communicating with the lumen are formed.

The catheter body portion 10 may form a layer structure so as to be laminated in the radial direction. The catheter body portion 10 includes an inner layer 11 disposed on an inner surface side, an outer layer 12 disposed on the outer periphery of the inner layer 11, a reinforcing material layer 13 disposed inside the outer layer 12, and a hydrophilic lubricating layer 14. In the form of FIG. 2, the inner layer 11 and the hydrophilic lubricating layer 14 are disposed to extend to the distal end of the catheter tip 20. Therefore, the inner layer 11 and the hydrophilic lubricating layer 14 are common to the inner layer and the hydrophilic lubricating layer in the catheter tip 20. In addition, in the catheter tip, the outer layer 12 (not including the inner layer 11 and the hydrophilic lubricating layer 14) may be formed of a single layer, or the outer layer 12 may be formed of a single layer and include a reinforcing material layer (metal reinforcing body layer) 13 in the outer layer 12, or the hydrophilic lubricating layer 14 may not be provided.

The inner layer 11 preferably includes a material that reduces friction at least at a portion that comes into contact with a device such as operating catheter and guidewire when the device is inserted through a lumen 10H. According to this configuration, the device inserted into the catheter body portion 10 is movable in a longitudinal direction with smaller sliding resistance, and operability can be improved. A specific example of the constituent material for the inner layer 11 includes a fluororesin material such as polytetrafluoroethylene (PTFE).

The outer layer 12 has a hollow tubular shape extending in the axial direction of the catheter body portion 10. Like the catheter body portion resin layer 12, the hydrophilic lubricating layer 14 has a hollow tubular shape extending in the axial direction of the catheter body portion 10. The outer layer 12 is preferably made of a material having kink resistance, suitable push-in property, and followability. Specifically, as the constituent material of the outer layer 12, polyamide elastomers and/or polyamides (polyamide elastomers, polyamides, or combinations of polyamide elastomers and/or polyamides), polyesters, polyester elastomers, polyurethane elastomers, polyurethanes, or combinations of the constituent materials listed above can be used.

The reinforcing material layer 13 includes a plurality of reinforcing wires for reinforcing the catheter body portion 10. Examples of the reinforcing wires include spiral or braided reinforcing wires. The reinforcing wire can be made of metal such as stainless steel. Specific examples of the reinforcing wire can include wires obtained by crushing stainless steel wires into a flat plate shape so that the thickness of the catheter body portion 10 in the radial direction becomes thin, and spiraling or weaving (braided body) the plurality of crushed wires of about 8 wires to 32 wires. The number of the reinforcing wires is preferably set to a multiple of 8 to implement reinforcement in a tubular shape in a well-balanced manner, but is not limited to the tubular shape in a well-balanced manner. Since the reinforcing wires uniformly receive an external stress by being formed into a flat plate compared to an ellipse, physical properties become constant.

The number of the layers constituting the catheter body portion 10 and the constituent material of each layer may be different along the longitudinal direction of the catheter body portion 10. For example, in order to make the portion on the distal side of the catheter body portion 10 more flexible, the number of the layers may be reduced, a more flexible material may be used, a reinforcing member may not be disposed only in the portion, or an inner layer may be provided up to the distal end.

When the hydrophilic lubricating layer 14 is provided, insertion into the living body becomes relatively smooth. The constituent material of the hydrophilic lubricating layer 14 is not particularly limited, and examples of the constituent material of the hydrophilic lubricating layer 14 can include copolymers of epoxy group-containing monomers such as glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether with hydrophilic monomers such as N-methylacrylamide, N,N-dimethylacrylamide, and acrylamide; (co) polymers containing the above-described hydrophilic monomer; cellulose-based polymer substances such as hydroxypropyl cellulose and carboxymethyl cellulose; polysaccharides, polyvinyl alcohol, methyl vinyl ether-maleic anhydride copolymers, water-soluble polyamides, poly(2-hydroxyethyl(meth)acrylate), polyethylene glycol, polyacrylamide, polyvinylpyrrolidone, and copolymers of polyvinylpyrrolidone and polyurethane described in U.S. Pat. No. 4,100,309 and Japanese Patent Application Publication No. S59-19582 A. These hydrophilic lubricating materials may be used singly or in combination of two or more kinds of the hydrophilic lubricating materials.

The hub 30 is mounted (fixed) on the proximal end of the catheter body portion 10. A lumen communicating with the lumen 10H and having a Luer taper is formed in the hub 30.

From the hub 30, for example, an elongated object (linear body) such as guidewire, catheter of any kind (for example, balloon catheter and stent delivery catheter), endoscope, ultrasonic probe, and temperature sensor is inserted or removed, and a liquid of any kind such as contrast medium (radiopaque medium), medicinal solution, or physiological saline is injected. In addition, for example, the hub 30 can also be connected to other instruments such as a Y-shaped branch connector. Furthermore, examples of a constituent material for the hub 30 can include a thermoplastic resin such as polycarbonate, polyamide, polysulfone, or polyarylate.

The catheter is inserted into the body while its position is confirmed under fluoroscopy. In the present disclosure, since the catheter tip 20 contains tungsten carbide as a radiopaque substance, the catheter body portion 10 does not necessarily contain the radiopaque substance, but the radiopaque substance (X-ray opaque substance) may be blended in the constituent material of the catheter body portion resin layer 12. As the radiopaque substance, for example, barium sulfate, bismuth oxide, tungsten, or the like can be used. The radiopaque substance is not limited to be present over the entire length of the catheter body portion 10, and may be present only in a part of the catheter body portion 10, for example, only in the distal portion of the catheter body portion 10 or only in the catheter tip 20.

Hereinafter, the catheter tip, the constituent material of the catheter tip, and the like will be described. In the present specification, a range from “X to Y” includes X and Y, and indicates “X or more and Y or less”. In addition, unless otherwise specified, operations and measurements of physical properties and the like are performed at room temperature (20° C. to 25° C.) and at relative humidity of 40% to 50% RH.

### Catheter Tip

The catheter tip 20 is formed of a material that is more flexible than the catheter body portion 10. The catheter tip 20 is also referred to as a distal tip, and has a function of suppressing damage to a lumen in a living body such as a blood vessel and a function of improving insertability into a stenosed part formed in the blood vessel. In FIG. 2, the catheter tip 20 has a tapered shape in which the outer diameter decreases toward the distal side, but the outer diameter may be substantially the same up to the distal side.

The catheter tip includes a catheter tip resin layer 21 containing a resin having an ester bond and/or an amide bond and tungsten carbide. Since the catheter tip contains tungsten carbide, the catheter tip has X-ray opaque property. The catheter tip resin layer may be a single layer or a plurality of layers of two or more layers. In the case of the plurality of layers, the compositions (resin types, mixing ratios, and the like) may be different or the same. Furthermore, the tungsten carbide is preferably dispersed in the resin as particles.

An elongation residual ratio represented by the above Formula (1) of the catheter tip resin layer may be 70% or more. By controlling the elongation residual ratio to 70% or more, durability for long-term storage can be further improved. The elongation residual ratio of the catheter tip resin layer is preferably 80% or more, and more preferably 85% or more (upper limit: 100%). As the elongation residual ratio of the catheter tip resin layer, a value measured by a method described in the following Examples is adopted. The elongation residual ratio can be controlled by appropriately selecting the content of the carbodiimide to be added, the type of the carbodiimide, and the like.

The strength residual ratio represented by the above Formula (2) of the catheter tip resin layer may be 60% or more. By controlling the strength residual ratio to 60% or more, durability for long-term storage can be further improved. The strength residual ratio of the catheter tip resin layer is preferably 70% or more, and more preferably 75% or more (upper limit: 100%). As the strength residual ratio of the catheter tip resin layer, a value measured by a method described in the following Examples is adopted. The strength residual ratio can be controlled by appropriately selecting the content of the carbodiimide to be added, the type of the carbodiimide, and the like.

The catheter tip may be composed of only the catheter tip resin layer, or another functional layer (the hydrophilic lubricating layer described above) may be laminated on the inner layer side (inner lumen side) and/or the outer layer side (outer surface side) of the catheter tip resin layer. The hydrophilic lubricating layer is as described above.

Resin Having Ester Bond and/or Amide Bond

Examples of the resin having an ester bond and/or an amide bond (hereinafter, also simply referred to as a resin having a bond) include a polyamide, a polyamide elastomer, a polyamideimide, a polyester, and a polyester elastomer. One kind of the resin may be used alone, or two or more kinds of resin may be laminated or blended.

Among the resins, since the resin having an ester bond and/or an amide bond has high flexibility and a small difference in affinity and hardness with an adjacent member, the resin preferably contains a polyamide-based resin, more preferably contains at least one of a polyamide and a polyamide elastomer, and still more preferably contains a polyamide elastomer. The content of the polyamide-based resin is preferably 50% by mass or more (upper limit: 100% by mass), more preferably 80% by mass or more, and still more preferably 100% by mass (composed of the polyamide-based resin), with respect to the total mass of the resin having an ester bond and/or an amide bond. The content of the polyamide elastomer is preferably 50% by mass or more (upper limit: 100% by mass), more preferably 80% by mass or more, and still more preferably 100% by mass (composed of the polyamide elastomer), with respect to the total mass of the resin having an ester bond and/or an amide bond.

In the resin constituting the catheter tip, the amount of the resin having an ester bond and/or an amide bond is preferably 80% by mass or more (upper limit: 100% by mass), more preferably 90% by mass or more, and still more preferably 100% by mass (composed of the resin having an ester bond and/or an amide bond), with respect to the total mass of the resin constituting the catheter tip.

The polyamide elastomer that can be used as the resin having an ester bond and/or an amide bond refers to a thermoplastic resin composed of a copolymer having a hard segment derived from a polymer that is crystalline and has a high melting point and a soft segment derived from a polymer that is amorphous and has a low glass transition temperature, the polyamide elastomer having amide bonds (—CONH—) in the polymer backbone forming the hard segment. Constituent units having amide bonds (—CONH—) in the polymer backbone forming the hard segment are also referred to as amide units of the polyamide elastomer.

The “amide units of the polyamide elastomer” in the present specification refers to repeating units derived from amide bonds in the polymer chain of the polyamide elastomer, and the amide units in the polyamide elastomer are preferably repeating units represented by Chemical formula (1) below.

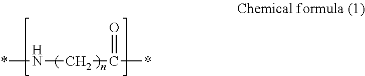

In the above Chemical formula (1), n is preferably an integer of 2 to 20, and more preferably an integer of 5 to 11. In Examples described later, n is 11. “*” is a bond to another repeating unit.

Examples of the polymer forming the soft segment include polyester and polyether. Moreover, examples of the polymer forming the soft segment can include polyethers such as polyethylene glycol, polypropylene glycol, polytetramethylene ether glycol (PTMG), and polyester polyol, and ABA-type triblock polyether diols. The polymer forming the soft segment may be used singly or in combination of two or more kinds of polymers. Furthermore, a polyether diamine or the like obtained by reacting ammonia or the like with the terminal of polyether can be used, and for example, an ABA-type triblock polyether diamine can be used. A polyether, which is a polymer capable of forming a soft segment, can form a polyether block amide copolymer bonded to a polyamide block as a hard segment, via an ester bond.

The content of the soft segment of the polyamide elastomer is preferably 1% to 90% by mass, and more preferably 10% to 70% by mass, with respect to the total mass of the polyamide elastomer.

The polyamide elastomer may also contain a polymer having a molecular weight increased by a chain extender such as a dicarboxylic acid with respect to a polymer composed of a hard segment and a soft segment.

These polyamide elastomers may be used singly or in combination of two or more kinds of polyamide elastomers.

The weight-average molecular weight of the polyamide elastomer is preferably 10,000 to 500,000, more preferably 15,000 to 400,000, and still more preferably 20,000 to 300,000. In one embodiment, the weight-average molecular weight of the polyamide elastomer is preferably 10,000 or more, more preferably 15,000 or more, and still more preferably 20,000 or more. In one embodiment, the weight-average molecular weight of the polyamide elastomer is preferably 500,000 or less, more preferably 300,000 or less, and still more preferably 200,000 or less.

The Shore D hardness of the polyamide elastomer used for the catheter tip is preferably 20 to 80, and more preferably 30 to 50 from the viewpoint of flexibility. In the present specification, as a method for measuring the hardness of the resin, for example, the polyamide elastomer, the Shore D hardness according to ISO 868:2003 is used. When plural kinds of polyamide elastomers are used in the present specification, the Shore D hardness of the whole polyamide elastomer in consideration of the content mass ratio is used.

The polyamide that can be used as the resin having an ester bond and/or an amide bond is not particularly limited as long as the polyamide is a polymer having an amide bond (—CO—NH—) in the backbone, and is usually produced by polymerization of a lactam or an amino acid having a ring structure or condensation polymerization of a dicarboxylic acid and a diamine. As the polyamide, a homopolyamide is preferably used. Examples of a homopolymerizable monomer include ε-caprolactam, ω-laurolactam, 6-aminocaproic acid, enanthlactam, 7-aminoheptanoic acid, 11-aminoundecanoic acid, 12-aminododecanoic acid, 9-aminononanoic acid, and piperidone.

In the condensation polymerization of a dicarboxylic acid and a diamine, examples of the dicarboxylic acid include adipic acid, sebacic acid, dodecane dicarboxylic acid, glutaric acid, terephthalic acid, 2-methylterephthalic acid, isophthalic acid, and naphthalenedicarboxylic acid. Examples of the diamine include tetramethylenediamine, hexamethylene diamine, nonamethylene diamine, decamethylene diamine, undecamethylene diamine, dodecamethylene diamine, p-phenylene diamine, and m-phenylenediamine.

Examples of the polyamide include NYLON 4, 6, 7, 8, 11, 12, 6.6, 6.9, 6.10, 6.11, 6.12, 6T, 6/6.6, 6/12, 6/6T, and 6T/61. The polyamide can be used singly or in combination of two or more kinds of polyamides.

The terminal of the polyamide may be capped with a carboxyl group, amine, or the like. Examples of the carboxylic acid include aliphatic monocarboxylic acids such as caproic acid, caprylic acid, capric acid, lauric acid, myristic acid, palmitic acid, stearic acid, and behenic acid. Furthermore, examples of the amine include aliphatic primary amines such as hexylamine, octylamine, decylamine, laurylamine, myristylamine, palmitylamine, stearylamine, and behenylamine.

The weight-average molecular weight of the polyamide is preferably 10,000 to 500,000, more preferably 15,000 to 400,000, and still more preferably 20,000 to 300,000.

The content of the resin in the catheter tip resin layer is appropriately set in consideration of X-ray contrast property, flexibility, moldability, and the like. As an example, when the content of tungsten carbide is 60% by mass or more with respect to the entire catheter tip resin layer, the content of the resin in the catheter tip resin layer is, for example, 10% to 40% by mass and may be 20% to 35% by mass. In one embodiment, the content of the resin in the catheter tip resin layer is preferably 10% by mass or more, and more preferably 20% by mass or more. In one embodiment, the content of the resin in the catheter tip resin layer is preferably 40% by mass or less, and more preferably 35% by mass or less.

As another example, when the content of tungsten carbide is less than 40% by mass with respect to the entire catheter tip resin layer, the content of the resin in the catheter tip resin layer is, for example, 60% to 99% by mass and may be 80% to 95% by mass.

In the present specification, when the catheter tip resin layer includes a plurality of layers, the content of a certain component in the catheter tip resin layer means the ratio of the total value of the masses of the certain components contained in all the catheter tip resin layers to the total value of the masses of all the catheter tip resin layers.

### Radiopaque Substance

The catheter tip resin layer also contains tungsten carbide as the radiopaque substance. As described above, the metal tungsten powder usually used as the radiopaque substance can release an acid by being hydrated after being oxidized, but the tungsten carbide is excellent because the tungsten carbide has almost no influence by being hydrated after being oxidized, and as a result, the risk of resin decomposition is reduced.

In one embodiment, only the tungsten carbide is used as the radiopaque substance. In one embodiment, the catheter tip resin layer contains a single metal tungsten (W) powder as the radiopaque substance. In one embodiment, the catheter tip resin layer does not contain a single metal tungsten (W). In one embodiment, the catheter tip contains a single metal tungsten (W) powder as the radiopaque substance. In one embodiment, the catheter tip does not contain a single metal tungsten (W). In the present specification, the term “not contain” means, for example, “not intentionally added as a raw material”, and a case of being contained due to unintended contamination, for example, degradation over time is also considered to be “not contained”. Specifically, the content of the metal tungsten (W) powder is preferably 0.01% by mass or less, and more preferably 0.001% by mass or less in the catheter tip resin layer.

The particle size of the tungsten carbide may be 1 μm to 10 μm. When the particle size of the tungsten carbide is 1 μm or more, the surface area is sufficiently small and the area being in contact with air is reduced, so that the decomposition, oxidation, and hydration reactions of the tungsten carbide hardly proceed, and as a result, the release of the acid is sufficiently suppressed. When the particle size of the tungsten carbide is 10 μm or less, the system tends to be uniform during mixing with the resin, thereby contributing to improvement of operability during producing and maintaining the high quality of the catheter tip resin layer. The particle size of the tungsten carbide is preferably 2 μm to 5 μm, and more preferably 3 μm to 4.5 μm. In one embodiment, the particle size of the tungsten carbide is 1 μm or more, preferably 2 μm or more, and more preferably 3 μm or more. In one embodiment, the particle size of the tungsten carbide is 10 μm or less, preferably 5 μm or less, and more preferably 4.5 μm or less. Here, in the present specification, the particle size refers to a volume average particle size, and can be measured by, for example, a dynamic light scattering method, but is not limited to the dynamic light scattering method.

Various tungsten carbides are commercially available, and in the present disclosure, these commercially available products can be preferably used.

The tungsten carbide may be contained in an amount of 60% by mass or more with respect to the entire catheter tip resin layer. When the content of the tungsten carbide is 60% by mass or more with respect to the entire catheter tip resin layer, the sufficient visibility of the catheter tip 20 can be secured during X-ray transmission. In one embodiment, the tungsten carbide is contained preferably in an amount of 60% to 80% by mass, more preferably in an amount of 65% to 80% by mass, and still more preferably in an amount of 70% to 80% by mass, with respect to the entire catheter tip resin layer. However, the tungsten carbide may be suitably contained in an amount of 60% to 70% by mass. In one embodiment, the tungsten carbide may be contained in an amount of 65% by mass or more, or 70% by mass or more with respect to the entire catheter tip resin layer. In one embodiment, the tungsten carbide may be contained in an amount of 80% by mass or less, or 70% by mass or less, with respect to the entire catheter tip resin layer.

A catheter tip having higher flexibility is required depending on the shape, fragility, and the like of an object using the catheter tip. In such a catheter tip, the tungsten carbide is preferably contained in a smaller amount. Therefore, in an embodiment of the present disclosure, the tungsten carbide may be contained in an amount of more than 0% by mass and less than 40% by mass with respect to the entire catheter tip resin layer. When the content of the tungsten carbide is more than 0% by mass with respect to the entire catheter tip resin layer, the catheter tip 20 is imaged during X-ray transmission. When the content of the tungsten carbide is less than 40% by mass, the catheter tip can have even more excellent flexibility. In one embodiment, the tungsten carbide is preferably contained in an amount of 1% to 20% by mass with respect to the entire catheter tip resin layer.

### Carbodiimide

The carbodiimide is a compound having a carbodiimide group (—N═C═N—) in the molecule. The carbodiimide group-containing compound is preferably polyfunctional (has two or more carbodiimide groups in the molecule).

The polyfunctional carbodiimide group-containing compound is preferably a cyclic carbodiimide or a polycarbodiimide. Among them, the polyfunctional carbodiimide group-containing compound is preferably a polycarbodiimide because the effect of the present disclosure is further exhibited.

Examples of the polycarbodiimide include an aromatic polycarbodiimide, an aliphatic polycarbodiimide, and an alicyclic polycarbodiimide. The aromatic polycarbodiimide is a polycarbodiimide which has an aromatic ring in the molecule and may or may not have an aliphatic ring. The aliphatic polycarbodiimide is a polycarbodiimide having no aromatic ring and no aliphatic ring in the molecule. The alicyclic polycarbodiimide is a polycarbodiimide having an aliphatic ring in the molecule but not having an aromatic ring. Among them, from the viewpoint of storage stability and the like, the aromatic polycarbodiimide and/or the alicyclic polycarbodiimide are preferable, the alicyclic polycarbodiimide is more preferable, and the polycarbodiimide may be cyclic.

Specific examples of the polycarbodiimide include a polycarbodiimide obtained by the decarboxylation condensation reaction of a diisocyanate. The diisocyanate may be, for example, any of a chain or alicyclic aliphatic diisocyanate compound, an aromatic diisocyanate compound, or a heterocyclic diisocyanate compound, and these may be used singly or in combination of two or more kinds of diisocyanate. Specifically, linear aliphatic diisocyanates such as tetramethylene diisocyanate, hexamethylene diisocyanate, dodecamethylene diisocyanate, and 2,2,4-trimethylhexamethylene diisocyanate; cycloaliphatic diisocyanates such as 1,4-bis(isocyanatomethyl)cyclohexane, 2,2-bis(4-isocyanatocyclohexyl) propane, isophorone diisocyanate, and dicyclohexylmethane-4,4′-diisocyanate; aliphatic diisocyanates containing aromatic rings such as 1,3-bis(2-isocyanato-2-propyl)benzene; and aromatic diisocyanates such as toluene-2,4-diisocyanate, 2,2′-diphenylmethane diisocyanate, 2,4′-diphenylmethane diisocyanate, 4,4′-diphenylmethane diisocyanate, and 2,4,6-triisopropylbenzene-1,3-diyl diisocyanate. These may be used singly or in combination of two or more kinds of linear aliphatic diisocyanates.

The polycarbodiimide is preferably one in which a terminal isocyanate group is sealed with a sealant from the viewpoint of storage stability. Examples of the sealant include a compound having active hydrogen that reacts with an isocyanate group or a compound having an isocyanate group. Examples of the sealant including a compound having active hydrogen that reacts with an isocyanate group or a compound having an isocyanate group include monoalcohols, monocarboxylic acids, monoamines, and monoisocyanates each having one substituent selected from a carboxy group, an amino group, and an isocyanate group.

The cyclic carbodiimide may include those having a macrocyclic structure in the whole molecule or a part of branches, and may particularly include a molecule in which a carbodiimide group (—N═C═N—) is introduced into the macrocyclic structure. Here, the macrocyclic structure can mean, for example, an 8-membered ring or more, a 10 membered ring or more, or a 12 membered ring or more. One, two, or three or more macrocyclic structures may be present in the molecule of the cyclic carbodiimide. The cyclic carbodiimide can be, for example, a compound of the Examples of the cyclic carbodiimide include a cyclic carbodiimide represented by Formula (i) (for example, the cyclic carbodiimide described on pages 13 to 15) as disclosed in International Publication No. WO 2010/071211 A and a cyclic carbodiimide represented by Formula (i) (specifically, the cyclic carbodiimide described on pages 10 to 11) as described in Japanese Patent Application Publication No. 2016-65001 A.

In one embodiment, the carbodiimide may be two or more kinds of carbodiimides. When two or more kinds of carbodiimides are used, both improvement of hydrolysis suppression ability and durability to temperature and humidity may be achieved. In particular, when two or more carbodiimides are contained, it is preferable to contain at least one of a polycarbodiimide and a cyclic carbodiimide, and it is more preferable to contain a polycarbodiimide and a cyclic carbodiimide.

In another embodiment, only one kind of carbodiimide may be used. When only one kind of carbodiimides is used, ease in the process of production is improved, and durability to temperature and humidity may be further improved as compared with the case of mixing two or more kinds of carbodiimides. In particular, when only one kind of carbodiimide is used, the carbodiimide is preferably a polycarbodiimide or a cyclic carbodiimide, and may be only a polycarbodiimide or only a cyclic carbodiimide.

A method for adding the carbodiimide to the resin for mixing is not particularly limited, and the carbodiimide and the resin may be mixed by dry blending, or the resin may be brought into a solution or a molten state, and then kneaded with the carbodiimide. A solvent may be used during kneading. As the solvent, for example, a hydrocarbon-based solvent, a ketone-based solvent, an ester-based solvent, an ether-based solvent, a halogen-based solvent, an amide-based solvent, or the like can be used.

The content of the carbodiimide is more than 0% by mass and 0.5% by mass or less with respect to the total mass of the catheter tip resin layer. The content of the carbodiimide is preferably more than 0% by mass and 0.3% by mass or less, more preferably 0.01% to 0.3% by mass, and still more preferably 0.05% to 0.3% by mass, with respect to the total mass of the catheter tip. When the addition amount of the hydrolysis inhibitor is within the above range, resin deterioration is effectively suppressed, and the characteristics of the resin are less likely to be influenced. The content of the carbodiimide may be, for example, more than 0% by mass and 0.25% by mass or less, more preferably 0.001% to 0.15% by mass, and still more preferably 0.005% to 0.15% by mass, with respect to the total mass of the resin in the catheter tip resin layer. When two or more carbodiimides are contained, the content of the carbodiimide represents the total value of the two or more carbodiimides.

In addition to the carbodiimide, other hydrolysis inhibitor may be added. As the other hydrolysis inhibitor, a conventionally known one can be used, and examples of the other hydrolysis inhibitor can include compounds that react with and bond to the carboxy group terminal of a degradation product. Specific examples of the other hydrolysis inhibitor include compounds containing functional groups such as an epoxy group (for example, a glycidyl ester compound, a glycidyl ether compound, and the like) and an oxazoline group (for example, a bisoxazoline compound and the like).

### HALS (Hindered Amine-Based Light Stabilizer)

A hindered amine-based light stabilizer (HALS) is often added to the catheter tip resin layer in order to secure long-term stability. However, since the addition of the HALS deteriorates the quality of the resin due to long-term storage, the addition of the HALS is preferably as small as possible. With the configuration of the present disclosure, the long-term stability is secured while the content of the HALS is reduced as much as possible.

Examples of the HALS (hindered amine-based light stabilizer) include, but are not limited to, bis(2,2,6,6-tetramethyl-4-piperidyl) sebacate, bis(1,2,2,6,6-pentamethyl-4-piperidyl) sebacate, and methyl 1,2,2,6,6-pentamethyl-4-piperidyl sebacate (mixture), bis(1,2,2,6,6-pentamethyl-4-piperidyl) [[3,5-bis(1,1-dimethylethyl)-4-hydrixyphenyl]methyl]butyl malonate, decanedioic acid bis(2,2,6,6-tetramethyl-1 (octyloxy)-4-piperidyl) ester and a reaction product of 1,1-dimethylethyl hydroperoxide and octane, 4-benzoyloxy-2,2,6,6-tetramethylpiperidine, an ester mixture of 2,2,6,6-tetramethyl-4-piperidinol and a higher fatty acid, tetrakis(2,2,6,6-tetramethyl-4-piperidyl)-1,2,3,4-butanetetracarboxylate, tetrakis(1,2,2,6,6-pentamethyl-4-piperidyl)-1,2,3,4-butanetetracarboxylate, a polycondensate of dimethyl succinate and 4-hydroxy-2,2,6,6-tetramethyl-1-piperidineethanol, poly[{(6-(1,1,3,3-tetramethylbutyl)amino-1,3,5-triazine-2,4-diyl) {(2,2,6,6-tetramethyl-4-piperidyl)imino}hexamethylene {(2,2,6,6-tetramethyl-4-piperidyl)imino}}], a polycondensate of dibutylamine·1,3,5-triazine·N,N′-bis(2,2,6,6-tetramethyl-4-piperidyl-1,6-hexamethylenediamine and N-(2,2,6,6-tetramethyl-4-piperidyl)butylamine, and N,N′, N″, N″-tetrakis-(4,6-bis-(butyl-(N-methyl-2,2,6,6-tetramethylpiperidin-4-yl)amino)-triazine-2-yl)-4,7-diazadecane-1,10-diamine. These HALS may be used singly or in combination of two or more kinds of HALS.

The content of the HALS may be more than 0% by mass and 0.1% by mass or less with respect to the total mass of the catheter tip resin layer. When the content of the HALS is more than 0% by mass, the deterioration of the resin caused by light can be satisfactorily suppressed, and when the content is 0.1% by mass or less, the coming up of the HALS to the surface of the resin can be suppressed. The catheter tip according to the present disclosure contains a carbodiimide and satisfactorily captures the generated acid (hydrogen ions). Therefore, since the generation of the acid (hydrogen ions) by light can be sufficiently suppressed, it is possible to sufficiently suppress the deterioration of the resin even when the content of the HALS is 0.1% by mass or less. In one embodiment, the content of the HALS is preferably more than 0% by mass and less than 0.1% by mass, more preferably more than 0% by mass and less than 0.05% by mass, and still more preferably 0.001% by mass or more and 0.025% by mass or less, with respect to the total mass of the catheter tip resin layer.

### Additive

In the present disclosure, the catheter tip resin layer can contain other additive. The additive that can be contained in the catheter tip resin layer is not particularly limited, and examples of the additive can include an ultraviolet absorber and an antioxidant.

### Ultraviolet Absorber

Examples of the ultraviolet absorber include, but are not limited to, benzotriazole-based ultraviolet absorbers such as 2-(2-hydroxy-5-methylphenyl)benzotriazole, 2-[2-hydroxy-3,5-bis(α,α-dimethylbenzyl)phenyl]benzotriazole, 2-(2-hydroxy-3-t-butyl-5-methylphenyl)-5-chlorobenzotriazole, 2-(2-hydroxy-3,5-di-t-butylphenyl)-5-chlorobenzotriazole, 2-(2-hydroxy-3,5-di-t-butylphenyl)benzotriazole, 2-(2-hydroxy-3,5-di-t-amylphenyl)benzotriazole, 2-(2-hydroxy-5-t-octylphenyl)benzotriazole, 2-(2-hydroxy-octoxyphenyl)benzotriazole, 2,2′-methylenebis[6-(2H-benzotriazole-2-yl)-4-(1,1,3,3-tetramethylbutyl)phenol], 2-[4,6-bis(2,4-dimethylphenyl)-1,3,5-triazine-2-yl]-5-(octyloxy)phenol, and 2-(4,6-diphenyl-1,3,5-triazine-2-yl)-5-(hexyloxy)phenol; benzophenone-based ultraviolet absorbers such as 2-hydroxy-4-octoxybenzophenone, 2-hydroxy-4-methoxybenzophenone, 2,4-dihydroxybenzophenone, and 2,2′,4,4′-tetrahydroxybenzophenone; and benzoate-based ultraviolet absorbers such as 2,4-di-t-butylphenyl 3,5-di-t-butyl-4-hydroxybenzoate and n-hexadecyl 3,5-di-t-butyl-4-hydroxybenzoate. These ultraviolet absorbers may be used singly or in combination of two or more kinds of ultraviolet absorbers.

The content of the ultraviolet absorber may be 0% by mass or more and less than 1.0% by mass with respect to the total mass of the catheter tip resin layer. When the content of the ultraviolet absorber is less than 1.0% by mass, the ultraviolet absorber can be suppressed from coming up to the surface of the resin. In one embodiment, the content of the ultraviolet absorber is preferably 0% by mass or more and 0.5% by mass or less, more preferably more than 0% by mass and 0.1% by mass or less, and still more preferably 0.001% by mass or more and 0.025% by mass or less, with respect to the total mass of the catheter tip resin layer.

### Antioxidant

In the present specification, the antioxidant can prevent deterioration such as denaturation or decomposition due to the oxidation of the resin. Examples of the antioxidant in the present specification include a phosphorus-based antioxidant, a sulfur-based antioxidant, and a phenol-based antioxidant. Specific examples of the phosphorus-based antioxidant include, but are not limited to, tris(nonylphenyl) phosphite, tris(2,4-di-t-butylphenyl) phosphite, distearyl pentaerythritol diphosphite, bis(2,4-di-t-butylphenyl)pentaerythritol diphosphite, bis(2,4-di-t-butyl-6-methylphenyl)pentaerythritol diphosphite, bis(2,6-di-t-butyl-4-methylphenyl)pentaerythritol diphosphite, bis(2,4-dicumylphenyl)pentaerythritol diphosphite, tetrakis(2,4-di-t-butylphenyl)-4,4′-diphenylenediphosphonite, 2,2′-methylenebis(4,6-di-t-butylphenyl) 2-ethylhexylphosphite, 2,2′-ethylidenebis(4,6-di-t-butylphenyl) fluorophosphite, bis(2,4-di-t-butyl-6-methylphenyl)ethyl phosphite, 2-(2,4,6-tri-t-butylphenyl)-5-ethyl-5-butyl-1,3,2-oxaphosphorinane, 2,2′,2″-nitrilotriethyl-tris(3,3′,5,5′-tetra-t-butyl-1, 1′-biphenyl-2,2′-diyl)phosphite, and 2,4,8, 10-tetra-t-butyl-6-[3-(3-methyl-4-hydroxy-5-t-butylphenyl)propoxy]dibenzo[d,f][1,3,2]dioxaphosphepine. Specific examples of the sulfur-based antioxidant include, but are not limited to, dilauryl-3,3′-thiopropionate, dimyristyl-3,3′-thiodipropionate, distearyl-3,3′-thiodipropionate, pentaerythritol tetrakis(3-laurylthiopropionate), ditridecyl-3,3′-thiodipropionate, and 1,3,5-tris-β-stearylthiopropionyloxyethyl isocyanurate. Specific examples of the phenolic antioxidant include, but are not limited to, 2,6-di-t-butyl-4-methylphenol, octadecyl-3-(3,5-di-t-butyl-4-hydroxyphenyl) propionate, pentaerythritol tetrakis[3-(3,5-di-t-butyl-4-hydroxyphenyl) propionate], 4,4′-butylidenebis(3-methyl-6-t-butylphenol), ethylenebis(oxyethylene)bis[3-(5-t-butyl-4-hydroxy-m-tolyl) propionate], 4,4′-thiobis(3-methyl-6-t-butylphenol), 1,3,5-trimethyl-2,4,6-tris(3,5-di-t-butyl-4-hydroxybenzyl)benzene, 1,3,5-tris(4-t-butyl-3-hydroxy-2,6-dimethylbenzyl) isocyanurate, 3,9-bis-{2-[3-(3-t-butyl-4 hydroxy-5-methylphenyl) propionyloxy]-1, 1-dimethylethyl}-2,4,8,10-tetraoxaspiro[5,5]undecane, tris-(3,5-di-t-butyl-4-hydroxybenzyl) isocyanurate, 2,2′-methylenebis(4-methyl-6-cyclohexylphenol), and 2,2′-dihydroxy-3,3′-di(α-methylcyclohexyl)-5,5′-dimethyldiphenylmethane. These antioxidants may be used singly or in combination of two or more kinds of antioxidants.

The content of the antioxidant may be 0% by mass or more and 1% by mass or less with respect to the total mass of the catheter resin layer. When the total value of the contents of the antioxidants is within the above range, the antioxidants can be suppressed from coming up to the surface of the resin. In one embodiment, the total value of the contents of the antioxidant and the light stabilizer is preferably 0% by mass or more and less than 1% by mass, more preferably more than 0% by mass and 0.5% by mass or less, still more preferably 0.001% by mass or more and 0.3% by mass or less, and yet still more preferably 0.001% by mass or more and 0.1% by mass or less, with respect to the total mass of the catheter tip resin layer.

The total value of the contents of the antioxidant and the light stabilizer may be more than 0% by mass and 3% by mass or less with respect to the total mass of the catheter resin layer. When the total value of the contents of the antioxidant and the light stabilizer is within the above range, the antioxidant and the light stabilizer can be suppressed from coming up to the surface of the resin. In one embodiment, the total value of the contents of the antioxidant and the light stabilizer may be 3% by mass or less, 1% by mass or less, 0.5% by mass or less, 0.1% by mass or less, 0.05% by mass or less, 0.02% by mass or less, or 0.01% by mass or less, with respect to the total mass of the catheter resin layer.

### Other Additives

The catheter tip resin layer may also contain other additives other than the antioxidant and the light stabilizer. Such additives can impart advantageous physical properties in the operability of the catheter tip and the like. In the present specification, examples of the other additives include, but are not limited to, a plasticizer, a lubricant, a surfactant, an antistatic agent, a filler, a diluent, a coupling agent, a colorant, a flame retardant, and a foaming agent.

Examples of the plasticizer include, but are not limited to, benzoic acid esters such as diethylene glycol dibenzoate, phthalic acid esters such as dibutyl phthalate (DBP), di-2-ethylhexyl phthalate (DOP), diisononyl phthalate (DINP), diisodecyl phthalate (DIDP), diundecyl phthalate (DUP), ditridecyl phthalate (DTDP), bis(2-ethylhexyl) terephthalate (DOTP), and bis(2-ethylhexyl) isophthalate (DOIP), aliphatic dibasic acid esters such as di-2-ethylhexyl adipate (DOA), diisononyl adipate (DINA), diisodecyl adipate (DIDA), di-2-ethylhexyl sebacate (DOS), and diisononyl sebacate (DINS), trimellitic acid esters such as tri-2-ethylhexyl trimellitate (TOTM), triisononyl trimellitate (TINTM), and triisodecyl trimellitate (TIDTM), pyromellitic acid esters such as tetra-2-ethylhexyl pyromellitate (TOPM), phosphoric acid esters such as tri-2-ethylhexyl phosphate (TOP) and tricresyl phosphate (TCP), alkyl esters of polyhydric alcohols such as pentaerythritol, epoxidized vegetable oils such as epoxidized soybean oil and epoxidized linseed oil, epoxy esters such as 4,5-epoxy-1,2-cyclohexanedicarboxylic acid di-2-ethylhexyl ester, alicyclic dibasic acid esters such as 1,2-cyclohexanedicarboxylic acid diisonyl (DINCH) and 4-cyclohexene-1,2-dicarboxylic acid di-2-ethylhexyl ester, fatty acid glycol esters such as 1,4-butanediol dicaprate, citric acid esters such as tributyl acetylcitrate (ATBC), trihexyl acetylcitrate (ATHC), triethylhexyl acetylcitrate (ATEHC), and trihexyl butyrylcitrate (BTHC), isosorbide diesters, chlorinated paraffins obtained by chlorinating paraffin wax and n-paraffin, chlorinated fatty acid esters such as chlorinated stearic acid ester, and higher fatty acid esters such as butyl oleate.

Examples of the lubricant include, but are not limited to, silicone, liquid paraffin, paraffin wax, fatty acid metal salts such as metal stearate and metal laurate, fatty acid amides, fatty acid wax, and higher fatty acid wax.

Examples of the surfactant include, but are not limited to, fatty acid esters of glycerin such as glycerin monostearate, glycerin monolaurate, glycerin monooleate, and glycerin monopalmitate; fatty acid esters of propylene glycol such as propylene glycol monolaurate, propylene glycol monostearate, propylene glycol monooleate, and propylene glycol monopalmitate; and fatty acid esters of sorbitan such as sorbitan monolaurate, sorbitan monostearate, sorbitan monoolate, and sorbitan monopalmitate.

Examples of the antistatic agent include, but are not limited to, an anionic antistatic agent of an alkyl sulfonate type, an alkyl ether carboxylic acid type, or a dialkyl sulfosuccinate type; a nonionic antistatic agent such as a polyethylene glycol derivative, a sorbitan derivative, or a diethanolamine derivative; a quaternary ammonium salt such as an alkylamidoamine type or an alkyldimethylbenzyl type; a cationic antistatic agent such as an organic acid salt or a hydrochloride salt of an alkylpyridinium type; and an amphoteric antistatic agent such as an alkylbetaine type or an alkylimidazoline type.

Examples of the diluent include, but are not limited to, 2,2,4-trimethyl-1,3-pentanediol diisobutyrate and low-boiling aliphatic and aromatic hydrocarbons.

Examples of the filler include, but are not limited to, metal oxides such as calcium carbonate, silica, alumina, clay, talc, diatomaceous earth, and ferrite, fibers and powders made of glass, carbon, and metals and the like, glass spheres, graphite, aluminum hydroxide, barium sulfate, magnesium oxide, magnesium carbonate, magnesium silicate, and calcium silicate.

Examples of the colorant include, but are not limited to, carbon black, lead sulfide, white carbon, titanium white, lithopone, red iron oxide, antimony sulfide, chromium yellow, chromium green, phthalocyanine green, cobalt blue, phthalocyanine blue, and molybdenum orange.

Examples of the flame retardant include, but are not limited to, inorganic compounds such as aluminum hydroxide, antimony trioxide, magnesium hydroxide, and zinc borate, phosphorus compounds such as cresyl diphenyl phosphate, tris chloroethyl phosphate, tris chloropropyl phosphate, and tris dichloropropyl phosphate, and halogen compounds such as chlorinated paraffin.

Examples of the foaming agent include, but are not limited to, organic foaming agents such as azodicarbonamide and oxybisbenzenesulfonyl hydrazide, and inorganic foaming agents such as sodium bicarbonate.

In addition to the catheter tip resin layer, the catheter tip may be provided with an inner layer such that at least a portion in contact with a device such as a treatment catheter or a guide wire when the device is inserted into the lumen 10H has low friction (inner layer 11 in FIG. 2). The inner layer is as described above.

### Catheter

The catheter 1 of the present disclosure is an elongated medical device that can be penetrated into a living body organ and used for the purpose of treating and improving a stenosed part or can be used for the purpose of delivering a medical instrument such as a stent in a living body. Examples of the living body organ include, but are not limited to, blood vessels, bile ducts, trachea, esophagus, other digestive tract, urethra, ear and nose lumen, and other organs. In one embodiment, the living body organ is a blood vessel, and for example, may be a coronary artery.

The catheter 1 of the present disclosure contains a radiopaque substance. As a result, when the catheter 1 is guided to a target location through the blood vessel, the catheter 1 can be visualized during X-ray irradiation by a contrast technique such as X-ray fluoroscopy. The radiopaque substance may be contained particularly in the catheter tip 20.

In one embodiment, the catheter 1 is used with an introducer sheath. In one embodiment, the catheter 1 is used with an introducer sheath and a guidewire. Here, the introducer sheath is a tubular medical device that penetrates a catheter from outside a living body into a blood vessel in advance in order to insert the catheter into the blood vessel, and the guide wire is a medical device that is introduced before the insertion of the catheter in order to assist in guiding the catheter to a desired location in the blood vessel. For example, after the introducer sheath is penetrated, the guide wire is penetrated through the introducer sheath, and the catheter 1 is inserted into the blood vessel up to the vicinity of a position where the catheter 1 is to be introduced. Thereafter, the catheter 1 is introduced into the blood vessel through the guide wire through a through hole inside the introducer sheath, and then guided along the guide wire through the blood vessel to a target location. Then, the guide wire is removed, whereby the catheter 1 can guide the medical device or the like to the target location.

Furthermore, the catheter according to the present embodiment may also include a guiding catheter and a catheter used as a guiding sheath.

### EXAMPLES

The effects of the present disclosure will be described with reference to the following Examples and Comparative Examples. Note that the technical scope of the present disclosure is not limited only to the following Examples. In the following Examples, unless otherwise specified, each operation was performed at room temperature (20° C. to 25° C.) and 40% to 50% RH.

### Preparation of Dumbbell-Shaped Test Piece

### Example 1

29.9 parts by mass of a polyamide elastomer (Shore D hardness: 40), 69.8 parts by mass of tungsten carbide particles (average particle size: 3.0 μm to 4.19 μm), 0.3 parts by mass of an alicyclic polycarbodiimide compound which was a carbodiimide group-containing compound as a hydrolysis inhibitor, and 0.019 parts by mass of a hindered amine-based light stabilizer (HALS; Tinuvin770) as a light stabilizer were weighed and mixed. The obtained mixture was press-molded at a set temperature of 200° C. using a 20 t (ton) manual hydraulic heating press. The thickness of the molded article was 1.9 mm to 2.1 mm. The molded press sheet was punched according to JIS K 7161-2:2014 5A to prepare a dumbbell-shaped test piece.

### Example 2

A dumbbell-shaped test piece of the present Example was prepared in the same manner as in Example 1 except that the content of a polyamide elastomer was 29.8 parts by mass, the content of tungsten carbide particles was 70 parts by mass, and the content of a carbodiimide group-containing compound (alicyclic polycarbodiimide compound) was 0.2 parts by mass.

### Example 3

A dumbbell-shaped test piece of the present Example was prepared in the same manner as in Example 1 except that the content of a polyamide elastomer was 29.95 parts by mass, the content of tungsten carbide particles was 69.9 parts by mass, a cyclic carbodiimide compound was used as a carbodiimide group-containing compound, and the content of the carbodiimide group-containing compound was 0.15 parts by mass.

### Example 4

A dumbbell-shaped test piece of the present Example was prepared in the same manner as in Example 1 except that the content of a polyamide elastomer was 29.7 parts by mass, the content of tungsten carbide particles was 70 parts by mass, an alicyclic polycarbodiimide compound and a cyclic carbodiimide compound were used as a carbodiimide group-containing compound, the content of the alicyclic polycarbodiimide compound was 0.15 parts by mass, and the content of the cyclic carbodiimide compound was 0.15 parts by mass.

### Comparative Example 1

A dumbbell-shaped test piece of the present Comparative Example was prepared in the same manner as in Example 1 except that the content of a polyamide elastomer was 30 parts by mass, the content of tungsten carbide particles was 70 parts by mass, and a carbodiimide group-containing compound was not added.

### Comparative Example 2

A dumbbell-shaped test piece of the present Comparative Example was prepared in the same manner as in Example 1 except that the content of a polyamide elastomer was 29.9 parts by mass, and 69.8 parts by mass of metal tungsten was added instead of tungsten carbide particles.

### Comparative Example 3

A dumbbell-shaped test piece of the present Comparative Example was prepared in the same manner as in Comparative Example 2 except that the content of a polyamide elastomer was 29.9 parts by mass, the content of metal tungsten was 69.8 parts by mass, and a cyclic carbodiimide compound (0.3 parts by mass) was used as a carbodiimide group-containing compound.

### Comparative Example 4

A dumbbell-shaped test piece of the present Comparative Example was prepared in the same manner as in Comparative Example 2 except that the content of a polyamide elastomer was 30 parts by mass, the content of metal tungsten was 70 parts by mass, and a carbodiimide group-containing compound was not added.

### Load Test

In order to examine the long-term durability of each of the dumbbell-shaped test pieces prepared in Examples and Comparative Examples, a load test was performed by subjecting the dumbbell-shaped test piece to high-temperature and high-humidity conditions. Specifically, the dumbbell-shaped test piece was allowed to stand under the conditions of a temperature of 80° C. and a humidity of 95% RH. In Examples 1 to 4 and Comparative Example 1, a standing time was set to 30 hours. In Comparative Examples 2 to 4, a standing time was set to 21 hours.

### Tensile Test

Each of the dumbbell-shaped test pieces of Examples 1 to 4 and Comparative Examples 1 to 4 before and after the load test was subjected to a tensile test according to JIS K 7161-1:2014.

### Test Conditions

Distance between grippers: 50 mm
Distance between gauge lines: 20 mm
Test speed: 100 mm/min

As values indicating durability, an elongation residual ratio represented by the following Formula (1) and a strength residual ratio represented by Formula (2) were calculated. The higher the elongation residual ratio and the strength residual ratio, the higher the durability. Here, a breaking stroke length refers to a value obtained by subtracting a distance between gauge lines before the test from a distance between marked lines after breaking. The results of the elongation residual ratios in Examples 1 to 4 and Comparative Example 1 were shown in Table 1, and the results of the elongation residual ratios in Comparative Examples 2 to 4 were shown in Table 2.

$$Elongation residual ratio [ \% ] = B / A \times 100 \qquad Formula ( 1 )$$

A: breaking stroke length before load test [mm]
B: breaking stroke length after load test [mm]

$$Strength residual ratio [ \% ] = D / C \times 100 \qquad Formula ( 2 )$$

C: breaking strength before load test [kgf]
D: breaking strength after load test [kgf]

TABLE 1
Table 1 - Results of tensile tests of Examples 1 to 4 and Comparative
example 1 before and after load test (80° C., 95% RH, 30 hours)

| | Content (parts by mass) | | | | | | Elongation |
|---|---|---|---|---|---|---|---|
| | | Carbodiimide | | Contrast | | | residual |
| | | Alicyclic | Cyclic | medium | | HALS | ratio [%] |
| | Resin | polycarbodiimide | carbodiimide | W | WC | (Tinuvin770) | (B/A × 100) |
| Example 1 | 29.9 | 0.3 | 0 | 0 | 69.8 | 0.019 | 89.22 |
| Example 2 | 29.8 | 0.2 | 0 | 0 | 70 | 0.019 | 83.48 |
| Example 3 | 29.95 | 0 | 0.15 | 0 | 69.9 | 0.019 | 86.34 |
| Example 4 | 29.7 | 0.15 | 0.15 | 0 | 70 | 0.019 | 86.51 |
| Comparative | 30 | 0 | 0 | 0 | 70 | 0.019 | 10.61 |
| example 1 | | | | | | | |

TABLE 2
Table 2 - Results of tensile tests of Comparative examples
2 to 4 before and after load test (80° C., 95% RH, 21 hours)

| | Content (parts by mass) | | | | | | Elongation |
|---|---|---|---|---|---|---|---|
| | | Carbodiimide | | Contrast | | | residual |
| | | Alicyclic | Cyclic | medium | | HALS | ratio [%] |
| | Resin | polycarbodiimide | carbodiimide | W | WC | (Tinuvin770) | (B/A × 100) |
| Comparative | 29.9 | 0.3 | 0 | 69.8 | 0 | 0.019 | 30.61 |
| example 2 | | | | | | | |
| Comparative | 29.9 | 0 | 0.3 | 69.8 | 0 | 0.019 | 8.14 |
| example 3 | | | | | | | |
| Comparative | 30 | 0 | 0 | 70 | 0 | 0.019 | 5.69 |
| example 4 | | | | | | | |

As a result of measuring the strength residual ratio residual ratios of Examples 1 to 4 and Comparative Examples 1 to 4, the strength residual ratios of Examples 1 to 4 were all 60% or more, whereas the strength residual ratios of Comparative Examples 1 to 4 were all less than 60%. As a result of these tensile tests, each of the dumbbell-shaped test pieces of Examples 1 to 4 had both an elongation residual ratio and a strength residual ratio before and after the load test higher than those of the dumbbell-shaped test piece of Comparative Example 1 containing no carbodiimide. Each of Comparative Examples 2 to 4 containing metal tungsten instead of tungsten carbide had an elongation residual ratio and a strength residual ratio lower before and after the load test than those of each of the dumbbell-shaped test pieces of Examples 1 to 4 although the load time was short. From this result, the dumbbell-shaped test pieces of Examples 1 to 4 were confirmed to have high durability against the load test.

Regarding the kind of carbodiimide, Example 1 in which only one kind of carbodiimide was used had an elongation residual ratio higher than that of Example 4 in which two kinds of carbodiimides were combined.

The detailed description above describes embodiments of a catheter tip. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter tip comprising:
a catheter tip resin layer containing a resin having an ester bond and/or an amide bond, carbodiimide, and tungsten carbide, wherein a content of the carbodiimide is more than 0% by mass and less than 0.5% by mass with respect to a total mass of the catheter tip resin layer.

2. The catheter tip according to claim 1, wherein an elongation residual ratio represented by Formula (1) is 70% or more:
$$Elongation residual ratio [ \% ] = B / A \times 100 \qquad Formula ( 1 )$$
wherein A represents a breaking stroke length in a tensile test of the catheter tip resin layer before a load test, B represents a breaking stroke length in the tensile test of the catheter tip resin layer after the load test, and
the load test is performed by allowing a dumbbell-shaped test piece to stand for 30 hours under conditions of a temperature of 80° C. and a humidity of 95% RH.

3. The catheter tip according to claim 1, wherein the content of the carbodiimide is more than 0% by mass and 0.3% by mass or less with respect to the total mass of the catheter tip resin layer.

4. The catheter tip according to claim 3, wherein the carbodiimide is composed of only one kind of carbodiimide.

5. The catheter tip according to claim 1, further comprising:
a content of the tungsten carbide is 60% by mass or more with respect to the total mass of the catheter tip resin layer.

6. The catheter tip according to claim 1, further comprising:
a content of a hindered amine light stabilizer (HALS) is more than 0% by mass and 0.1% by mass or less with respect to the total mass of the catheter tip resin layer.

7. The catheter tip according to claim 1, wherein the elongation residual ratio is 80% or more.

8. The catheter tip according to claim 1, wherein a strength residual ratio represented by Formula (2) is 60% or more:
$$Strength residual ratio [ \% ] = D / C \times 100 \qquad Formula ( 2 )$$
wherein C represents a breaking strength in a tensile test of the catheter tip resin layer before the load test, and D represents a breaking strength in the tensile test of the catheter tip resin layer after a load test, wherein the load test is performed by allowing a dumbbell-shaped test piece to stand for 30 hours under conditions of a temperature of 80° C. and a humidity of 95% RH.

9. A catheter comprising the catheter tip according to claim 1.

10. The catheter according to claim 9, wherein the catheter is used as at least one of a guiding catheter and a guiding sheath.

11. A catheter comprising:
a catheter body portion extending in an axial direction;
a catheter tip disposed on a distal side of the catheter body portion;
a hub disposed on a proximal side of the catheter body portion; and
wherein the catheter includes a catheter tip resin layer containing a resin having an ester bond and/or an amide bond, carbodiimide, and tungsten carbide, wherein a content of the carbodiimide is more than 0% by mass and less than 0.5% by mass with respect to a total mass of the catheter tip resin layer.

12. The catheter according to claim 11, wherein the catheter tip has an elongation residual ratio represented by Formula (1) is 70% or more:
$$e longation residual ratio [ \% ] = B / A \times 100 \qquad Formula ( 1 )$$
wherein A represents a breaking stroke length in a tensile test of the catheter tip resin layer before a load test, B represents a breaking stroke length in the tensile test of the catheter tip resin layer after a load test, and
the load test is performed by allowing a dumbbell-shaped test piece to stand for 30 hours under conditions of a temperature of 80° C. and a humidity of 95% RH.

13. The catheter according to claim 11, wherein the content of the carbodiimide in the catheter tip is more than 0% by mass and 0.3% by mass or less with respect to the total mass of the catheter tip resin layer.

14. The catheter according to claim 13, wherein the carbodiimide of the catheter tip is composed of only one kind of carbodiimide.

15. The catheter according to claim 11, further comprising:
a content of the tungsten carbide of the catheter tip is 60% by mass or more with respect to the total mass of the catheter tip resin layer.

16. The catheter according to claim 11, wherein a content of a hindered amine light stabilizer (HALS) of the catheter tip is more than 0% by mass and 0.1% by mass or less with respect to the total mass of the catheter tip resin layer.

17. The catheter according to claim 11, wherein a strength residual ratio of the catheter tip is represented by the following Formula (2) is 60% or more:
$$Strength residual ratio [ \% ] = D / C \times 100 \qquad Formula ( 2 )$$
wherein C represents a breaking strength in a tensile test of the catheter tip resin layer before the load test, and D represents a breaking strength in the tensile test of the catheter tip resin layer after a load test, wherein the load test is performed by allowing a dumbbell-shaped test piece to stand for 30 hours under conditions of a temperature of 80° C. and a humidity of 95% RH.

18. The catheter according to claim 11, further comprising:
a kink-resistant protector disposed between the catheter body portion and the hub.

19. A catheter comprising:
a catheter body portion extending in an axial direction;
a catheter tip disposed on a distal side of the catheter body portion;
the catheter includes a catheter tip resin layer containing a resin having an ester bond and/or an amide bond, carbodiimide, and tungsten carbide, wherein a content of the carbodiimide is more than 0% by mass and less than 0.5% by mass with respect to a total mass of the catheter tip resin layer;
wherein the catheter tip has an elongation residual ratio represented by Formula (1) is 70% or more:
$$e longation residual ratio [ \% ] = B / A \times 100 \qquad Formula ( 1 )$$
wherein A represents a breaking stroke length in a tensile test of the catheter tip resin layer before a load test, B represents a breaking stroke length in the tensile test of the catheter tip resin layer after a load test, and
the load test is performed by allowing a dumbbell-shaped test piece to stand for 30 hours under conditions of a temperature of 80° C. and a humidity of 95% RH; and
wherein the content of the carbodiimide in the catheter tip is more than 0% by mass and 0.3% by mass or less with respect to the total mass of the catheter tip resin layer.

20. The catheter according to claim 19, further comprising:
a content of the tungsten carbide is 60% by mass or more with respect to the total mass of the catheter tip resin layer; and
a content of a hindered amine light stabilizer (HALS) is more than 0% by mass and 0.1% by mass or less with respect to the total mass of the catheter tip resin layer.
